(19) United States
(12) Patent Application Publication
(10) Pub. No.: US 2017/0145351 A1
(43) Pub. Date: May 25, 2017

(54) ACTIVE INGREDIENTS WHICH IMPROVE PRIMARY DETERGENT POWER

(71) Applicant: Henkel AG & Co. KGaA, Duesseldorf (DE)

(72) Inventors: Andre Haetzelt, Duesseldorf (DE); Thomas Gerke, Duesseldorf (DE); Juergen Schatz, Adelsdorf (DE); Silvia Onodi, Dietfurt (DE)

(73) Assignee: Henkel AG & Co. KGaA, Duesseldorf (DE)

(21) Appl. No.: 15/405,608

(22) Filed: Jan. 13, 2017

Related U.S. Application Data
(63) Continuation of application No. PCT/EP2015/065651, filed on Jul. 9, 2015.

(30) Foreign Application Priority Data
Jul. 17, 2014 (DE) 10 2014 010 505.9

(51) Int. Cl. C11D 3/34; C11D 11/00; C07C 323/21

(52) U.S. Cl. CPC C11D 3/34; C07C 323/21; C11D 11/0017; C11D 11/0023

(57) ABSTRACT

The aim of the invention is to improve the primary detergent power of washing and cleaning agents, in particular with respect to bleach-sensitive or enzyme-sensitive soiling. This is substantially achieved by the incorporation of alkoxy group-carrying sulfocalixarenes or carboxycalixarenes.

### FIELD OF THE INVENTION

The present invention generally relates to the use of certain macrocyclic oligomers of the calixarene type to enhance the primary detergent power of washing or cleaning agents when washing textiles or cleaning hard surfaces with respect to in particular bleach-sensitive or enzyme-sensitive soiling, and to washing and cleaning agents which contain such macrocyclic oligomers.

### BACKGROUND OF THE INVENTION

Besides the ingredients essential for the washing process, such as surfactants and builder materials, washing agents usually contain further constituents which can be collectively referred to as washing auxiliaries and which include such diverse groups of active substances as foam regulators, anti-redeposition agents, bleaching agents, bleach activators and dye transfer inhibitors. Such auxiliaries also include substances which, when present, enhance the detergent power of surfactants without generally themselves having a pronounced surfactant behavior. The same also applies to the ingredients of cleaning agents for hard surfaces. Such substances are often referred to as detergent power enhancers.

It has surprisingly been found that certain macrocyclic oligomers of the calixarene type have properties that particularly enhance the primary detergent power.

Furthermore, other desirable features and characteristics of the present invention will become apparent from the subsequent detailed description of the invention and the appended claims, taken in conjunction with this background of the invention.

### BRIEF SUMMARY OF THE INVENTION

The use of compounds of general formula (I)

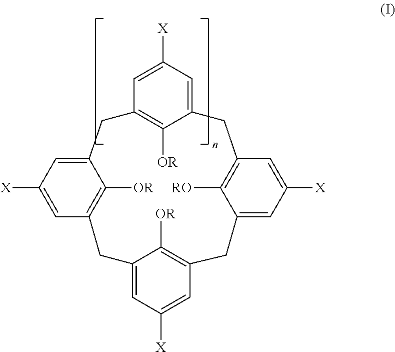

in which n represents a number from 1 to 9, R represents hydrogen or an alkyl group having 1 to 20 C atoms, X represents hydrogen, —Y—COOM or —Y—SO₃M, Y represents a direct bond, an alkylene group having 1 to 12 C atoms, an arylene or heteroarylene group having 2 to 12 C atoms, a mono- or poly-alkyl-substituted arylene group or a mono- or poly-aryl-substituted alkylene group having 6 to 18 C atoms, and M represents hydrogen, an alkali metal atom or an ammonium group, with the provisos that in the compound of general formula (I) not all substituents R need have the same meaning, that not all substituents X need have the same meaning, that not all groups Y need have the same meaning, that at least 1 substituent X is not hydrogen and that at least 1 substituent R is not hydrogen, for enhancing the primary detergent power of washing or cleaning agents when washing textiles or when cleaning hard surfaces with respect to in particular bleach-sensitive or enzyme-sensitive soiling.

A method for removing in particular bleach-sensitive or enzyme-sensitive soiling from textiles or hard surfaces, in which use is made of a washing or cleaning agent and a compound of general formula (I)

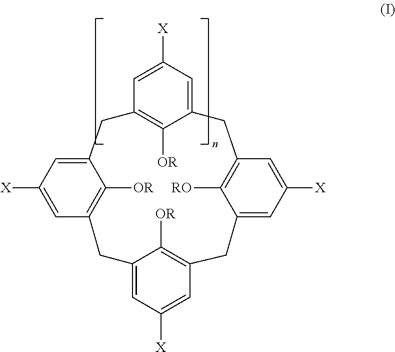

in which n represents a number from 1 to 9, R represents hydrogen or an alkyl group having 1 to 20 C atoms, X represents hydrogen, —Y—COOM or —Y—SO₃M, Y represents a direct bond, an alkylene group having 1 to 12 C atoms, an arylene or heteroarylene group having 2 to 12 C atoms, a mono- or poly-alkyl-substituted arylene group or a mono- or poly-aryl-substituted alkylene group having 6 to 18 C atoms, and M represents hydrogen, an alkali metal atom or an ammonium group, with the provisos that in the compound of general formula (I) not all substituents R need have the same meaning, that not all substituents X need have the same meaning, that not all groups Y need have the same meaning, that at least 1 substituent X is not hydrogen, and that at least 1 substituent R is not hydrogen.

A washing or cleaning agent, containing a compound of general formula (I)

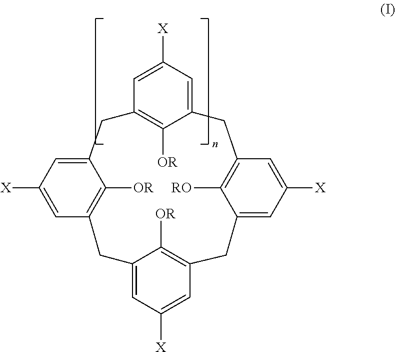

in which n represents a number from 1 to 9, R represents hydrogen or an alkyl group having 1 to 20 C atoms, X represents hydrogen, —Y—COOM or —Y—SO₃M, Y represents a direct bond, an alkylene group having 1 to 12 C atoms, an arylene or heteroarylene group having 2 to 12 C atoms, a mono- or poly-alkyl-substituted arylene group or a mono- or poly-aryl-substituted alkylene group having 6 to 18 C atoms, and M represents hydrogen, an alkali metal atom or an ammonium group, with the provisos that in the compound of general formula (I) not all substituents R need have the same meaning, that not all substituents X need have the same meaning, that not all groups Y need have the same meaning, that at least 1 substituent X is not hydrogen, and that at least 1 substituent R is not hydrogen.

### DETAILED DESCRIPTION OF THE INVENTION

The following detailed description of the invention is merely exemplary in nature and is not intended to limit the invention or the application and uses of the invention. Furthermore, there is no intention to be bound by any theory presented in the preceding background of the invention or the following detailed description of the invention.

The subject matter of the invention is the use of compounds of general formula (I)

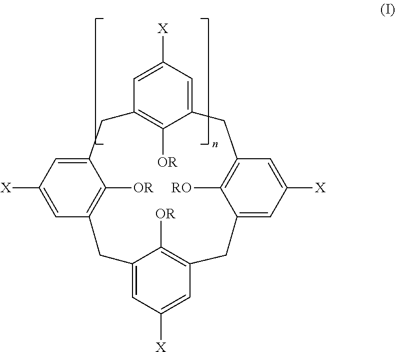

in which n represents a number from 1 to 9, in particular 3 to 7, R represents hydrogen or an alkyl group having 1 to 20 C atoms, in particular 6 to 12 C atoms and preferably 8 to 10 C atoms, X represents hydrogen, —Y—COOM or —Y—SO₃M, Y represents a direct bond, an alkylene group having 1 to 12, in particular 2 to 4 C atoms, an arylene or heteroarylene group having 2 to 12, in particular 2 to 6 C atoms, a mono- or poly-alkyl-substituted arylene group or a mono- or poly-aryl-substituted alkylene group having 6 to 18, in particular 6 to 12 C atoms, and M represents hydrogen, an alkali metal atom or an ammonium group, preferably sodium and/or potassium, with the provisos that in the compound of general formula (I) not all substituents R need have the same meaning, that not all substituents X need have the same meaning, that not all groups Y need have the same meaning, that at least 1 substituent X is not hydrogen, and that at least 1 substituent R is not hydrogen, for enhancing the primary detergent power of washing or cleaning agents when washing textiles or when cleaning hard surfaces with respect to in particular bleach-sensitive or enzyme-sensitive soiling.

The macrocyclic active substance of general formula (I) can be obtained by alkylation of commercially available sulfocalixarenes or carboxycalixarenes. The preferred compounds of general formula (I) include those in which n is 1, 3 or 5. The preferred compounds of general formula (I) also include those in which all substituents X are not hydrogen, particularly those in which all substituents X are COOM or SO₃M, and among these particular preference is given to those in which all substituents X are identical and/or M is hydrogen. The preferred compounds of general formula (I) also include those in which all substituents R are not hydrogen, particularly those in which all substituents R are identical.

Another subject matter of the invention is a method for removing in particular bleach-sensitive or enzyme-sensitive soiling from textiles or hard surfaces, in which use is made of a washing or cleaning agent and an aforementioned macrocyclic active substance. Bleach-sensitive or enzyme-sensitive soiling will be understood to mean soiling which can usually be at least partially removed by bleaching agents or with the aid of enzymes. This method may be carried out by hand or by a machine, for example by means of a domestic washing machine or dishwasher. It is possible to apply the agent and the active substance simultaneously or one after the other. Simultaneous application can be performed with particular advantage by using a washing or cleaning agent, in particular a liquid washing or cleaning agent, which contains the active substance.

Another subject matter of the invention is therefore a washing or cleaning agent, containing a compound of general formula (I)

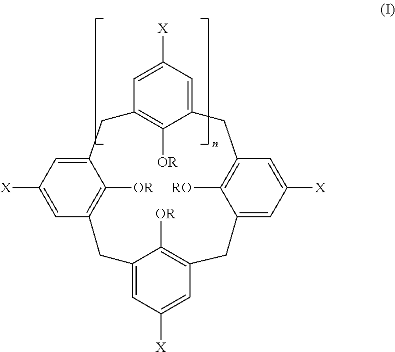

in which n represents a number from 1 to 9, in particular 3 to 7, R represents hydrogen or an alkyl group having 1 to 20 C atoms, in particular 6 to 12 C atoms and preferably 8 to 10 C atoms, X represents hydrogen, —Y—COOM or —Y—SO3M, Y represents a direct bond, an alkylene group having 1 to 12, in particular 2 to 4 C atoms, an arylene or heteroarylene group having 2 to 12, in particular 2 to 6 C atoms, a mono- or poly-alkyl-substituted arylene group or a mono- or poly-aryl-substituted alkylene group having 6 to 18, in particular 6 to 12 C atoms, and M represents hydrogen, an alkali metal atom or an ammonium group, preferably sodium and/or potassium, with the provisos that in the compound of general formula (I) not all substituents R need have the same meaning, that not all substituents X need have the same meaning, that not all groups Y need have the same meaning, that at least 1 substituent X is not hydrogen, and that at least 1 substituent R is not hydrogen.

The compounds of general formula (I) which are preferred in the context of the use aspect of the invention are also preferred in the aspects of the invention relating to the method and the washing and cleaning agent.

The use of the active substances used according to the invention leads to a significantly better removal of in particular bleach-sensitive or enzyme-sensitive soiling on hard surfaces and on textiles, including those made of cotton or containing cotton, than is the case when using compounds previously known for this purpose. Alternatively, for the same soil removal capability, significant quantities of surfactants can be saved. It is particularly noteworthy that the enhancing of the detergent power when washing textiles occurs even at low temperatures. One preferred embodiment of the invention therefore relates to the use of said active substance at temperatures of 20° C. and below, particularly when washing textiles.

The use according to the invention may take place in the context of a washing or cleaning process in such a way that the compound of general formula (I) is added to a liquor containing washing or cleaning agent or preferably the compound of general formula (I) is introduced into the liquor as a constituent of a washing or cleaning agent, wherein the concentration of the compound of general formula (I) in liquor containing washing or cleaning agent is preferably up to 5 mmol/l and in particular lies in the range from 0.1 mmol/l to 2.5 mmol/l.

Washing or cleaning agents which contain an active substance to be used according to the invention or which are used together with said active substance or are used in the method according to the invention may contain all the usual other constituents of such agents, which do not interact undesirably with the active substance essential to the invention. Preferably, an active substance defined above is incorporated in washing or cleaning agents in amounts of 0.1% by weight to 10% by weight, in particular 0.5% by weight to 2% by weight.

It has surprisingly been found that such active substances have a positive effect on the action of certain other washing and cleaning agent ingredients and that, conversely, the effect of the active substance is additionally further enhanced by certain other ingredients. These effects occur in particular with the ingredients alkylbenzene sulfonate, ethoxylated fatty alcohol, sulfated fatty alcohol ethoxylate and fatty alkyl amine N-oxide and mixtures thereof, and therefore preference is given to the use of at least one, preferably at least two, in particular at least three and particularly preferably four of the aforementioned further ingredients together with the active substance to be used according to the invention.

An agent which contains an active substance to be used according to the invention or which is used together with said active substance or which is used in the method according to the invention may preferably contain peroxygen-based bleaching agents, in particular in amounts ranging from 5% by weight to 70% by weight, and also optionally bleach activators, in particular in amounts ranging from 0.3% by weight to 10% by weight. Suitable bleaching agents are preferably the peroxygen compounds usually used in washing agents, such as percarboxylic acids, for example dodecanediperoic acid or phthaloylimidoperoxycaproic acid, hydrogen peroxide, alkali perborate, which may be in the form of the tetra- or monohydrate, percarbonate, perpyrophosphate and persilicate, which are usually in the form of alkali salts, in particular sodium salts. Such bleaching agents are present in agents which contain an active substance used according to the invention preferably in amounts up to 25% by weight, in particular up to 15% by weight and particularly preferably from 5% by weight to 15% by weight, in each case based on the total agent, percarbonate being used in particular.

As a bleach activator compound which under perhydrolysis conditions yields peroxocarboxylic acid, use may be made in particular of compounds which under perhydrolysis conditions yield optionally substituted perbenzoic acid and/or aliphatic peroxocarboxylic acids having 1 to 12 C atoms, in particular 2 to 4 C atoms, alone or in mixtures. Bleach activators which carry O- and/or N-acyl groups, in particular of the aforementioned number of C atoms, and/or optionally substituted benzoyl groups are suitable. Preference is given to polyacylated alkylenediamines, in particular tetraacetylethylenediamine (TAED), acylated glycolurils, in particular tetraacetyl glycoluril (TAGU), acylated triazine derivatives, in particular 1,5-diacetyl-2,4-dioxohexahydro-1,3,5-triazine (DADHT), N-acylimides, in particular N-nonanoyl succinimide (NOSI), acylated phenolsulfonates or -carboxylates or the sulfonic acids or carboxylic acids thereof, in particular nonanoyl or isononanoyl or lauroyl oxybenzenesulfonate (NOBS or iso-NOBS or LOBS), or decanoyloxybenzoate (DOBA), formal carbonic acid ester derivatives thereof such as 4-(2-decanoyloxyethoxycarbonyloxy)benzenesulfonate (DECOBS), acylated polyvalent alcohols, in particular triacetin, ethylene glycol diacetate and 2,5-diacetoxy-2,5-dihydrofuran, as well as acetylated sorbitol and mannitol and mixtures thereof (SORMAN), acylated sugar derivatives, in particular pentaacetylglucose (PAG), pentaacetylfructose, tetraacetylxylose and octaacetyllactose, acetylated, optionally N-alkylated glucamine and gluconolactone, and/or N-acylated lactams, for example N-benzoylcaprolactam.

In addition to or as an alternative to the compounds which under perhydrolysis conditions form peroxocarboxylic acids, further bleach activator compounds may be present, such as for example nitriles from which perimidic acids are formed under perhydrolysis conditions. These include in particular aminoacetonitrile derivatives having a quaternized nitrogen atom according to the formula

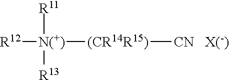

in which R¹¹ represents —H, —CH₃, a C₂₋₂₄ alkyl or alkenyl radical, a substituted C₁₋₂₄ alkyl or C₂₋₂₄ alkenyl radical having at least one substituent from the group —Cl, —Br, —OH, —NH₂, —CN and —N⁽⁺⁾—CH₂—CN, an alkyl or alkenylaryl radical having a C₁₋₂₄ alkyl group, or a substituted alkyl or alkenylaryl radical having at least one, preferably two, optionally substituted C₁₋₂₄ alkyl group(s) and optionally further substituents on the aromatic ring, R¹² and R¹³ independently of one another are selected from —CH₂—CN, —CH₃, —CH₂—CH₃, —CH₂—CH₂—CH₃, —CH(CH₃)—CH₃, —CH₂—OH, —CH₂—CH₂—OH, —CH(OH)—CH₃, —CH₂—CH₂—CH₂—OH, —CH₂—CH(OH)—CH₃, —CH(OH)—CH₂—CH₃, —(CH₂CH₂—O)ₙH where n=1, 2, 3, 4, 5 or 6, R¹⁴ and R¹⁵ independently of one another have a meaning specified above for R¹¹, R¹² or R¹³, wherein at least 2 of the aforementioned radicals, in particular R¹² and R¹³, may also be linked to one another to close the ring with inclusion of the nitrogen atom and optionally of further heteroatoms, and then preferably form a morpholino ring, and X is a charge-compensating anion, preferably selected from benzenesulfonate, toluenesulfonate, cumenesulfonate, C₉₋₁₅ alkylbenzenesulfonates, C₁₋₂₀ alkyl sulfates, C₈₋₂₂ carboxylic acid methyl ester sulfonates, sulfate, hydrogen sulfate, and mixtures thereof. Oxygen-transferring sulfonimines can also be used.

Bleach activators are contained in washing or cleaning agents preferably in amounts of up to 8% by weight, in particular from 2% by weight to 6% by weight, in each case based on the total agent.

To avoid interaction with the peroxygen compounds during storage, the bleach activators may in a known manner have been coated with enveloping substances or may have been granulated, particular preference being given to tetraacetylethylenediamine granulated with carboxymethylcellulose and having average particle sizes of 0.01 mm to 0.8 mm, granulated 1,5-diacetyl-2,4-dioxohexahydro-1,3,5-triazine, and/or trialkylammonium acetonitrile prepared in particle form.

In addition to such bleach activators or if desired in place thereof, use may also be made of conventional transition metal complexes which activate the bleach. These are preferably selected from cobalt, iron, copper, titanium, vanadium, manganese and ruthenium complexes. Suitable ligands in such transition metal complexes are both inorganic and organic compounds, among which are included, besides carboxylates, in particular compounds having primary, secondary and/or tertiary amine and/or alcohol functions, such as pyridine, pyridazine, pyrimidine, pyrazine, imidazole, pyrazole, triazole, 2,2′-bispyridylamine, tris-(2-pyridylmethyl)amine, 1,4,7-triazacyclononane and substituted derivatives thereof such as 1,4,7-trimethyl-1,4,7-triazacyclononane, 1,5,9-triazacyclododecane and substituted derivatives thereof such as 1,5,9-trimethyl-1,5,9-triazacyclododecane, 1,4,8,11-tetraazacyclotetradecane and substituted derivatives thereof such as 5,5,7,12,12,14-hexamethyl-1,4,8,11-tetraazacyclotetradecane, 1,5,8,12-tetraazabicyclo[6.6.2]hexadecane and substituted derivatives thereof such as 5,12-diethyl-1,5,8,12-tetraazabicyclo[6.6.2]hexadecane, (bis-(1-methylimidazol-2-yl)methyl))-(2-pyridylmethyl)amine, N,N′-(bis-(1-methylimidazol-2-yl)methyl)ethylenediamine, N-bis-(2-benzimidazolylmethyl)amino-ethanol, 2,6-bis-(bis-(2-benzimidazolylmethyl)aminomethyl)-4-methylphenol, N,N,N′,N′-tetrakis-(2-benzimidazolylmethyl)-2-hydroxy-1,3-diaminopropane, 2,6-bis-(bis-(2-pyridylmethyl)aminomethyl)-4-methylphenol, 1,3-bis-(bis-(2-benzimidazolylmethyl)aminomethyl)benzene, sorbitol, mannitol, erythritol, adonitol, inositol, lactose, and optionally substituted salens, porphins and porphyrins. The inorganic neutral ligands include in particular ammonia and water. If not all the coordination sites of the transition metal central atom are occupied by neutral ligands, the complex contains further, preferably anionic, and among the latter in particular unidentate or bidentate ligands. These include in particular halides such as fluoride, chloride, bromide and iodide, and the (NO₂) group, that is to say a nitro ligand or a nitrilo ligand. The (NO₂) group may also be bound to a transition metal to form a chelate, or it can bridge two transition metal atoms asymmetrically or in η¹-O fashion. Besides the aforementioned ligands, the transition metal complexes may also carry further ligands which are generally of simpler construction, in particular mono- or polyvalent anionic ligands. Nitrate, acetate, trifluoracetate, formate, carbonate, citrate, oxalate, perchlorate and complex anions such as hexafluorophosphate are suitable for example. The anionic ligands are intended to provide charge compensation between the transition metal central atom and the ligand system. The presence of oxo ligands, peroxo ligands and imino ligands is also possible. In particular, such ligands may also act in bridging fashion, so that polynuclear complexes are produced. In the case of bridged binuclear complexes, the two metal atoms in the complex need not be identical. The use of binuclear complexes in which the two transition metal central atoms have different oxidation numbers is also possible. If anionic ligands are absent, or if the presence of anionic ligands does not lead to charge compensation in the complex, anionic counter-ions which neutralize the cationic transition metal complex are then present in the transition metal complex compounds to be used according to the invention. These anionic counter-ions include in particular nitrate, hydroxide, hexafluorophosphate, sulfate, chlorate, perchlorate, halides such as chloride, or anions of carboxylic acids such as formate, acetate, oxalate, benzoate or citrate. Examples of transition metal complex compounds which can be used are Mn(IV)₂(μ-O)₃(1,4,7-trimethyl-1,4,7-triazacyclononane)dihexafluorophosphate, [N,N′-bis[(2-hydroxy-5-vinylphenyl)methylene]-1,2-diaminocyclohexane]manganese(111) chloride, [N,N′-bis[(2-hydroxy-5-nitrophenyl)methylene]-1,2-diaminocyclohexane]manganese(111) acetate, [N,N′-bis[(2-hydroxyphenyl)methylene]-1,2-phenylenediamine]manganese(111) acetate, [N,N′-bis[(2-hydroxyphenyl)methylene]-1,2-diaminocyclohexane]manganese(111) chloride, [N,N′-bis[(2-hydroxyphenyl)methylene]-1,2-diaminoethane]manganese(111) chloride, [N,N′-bis[(2-hydroxy-5-sulfonatophenyl)methylene]-1,2-diaminoethane]manganese(III) chloride, manganese-oxalato complexes, nitropentaamminecobalt(111) chloride, nitritopentaamminecobalt(111) chloride, hexamminecobalt(111) chloride, chloropentamminecobalt(111) chloride, and the peroxo complex [(NH₃)₅Co—O—O—Co(NH₃)₅]Cl₄.

Bleach-activating transition metal complexes are contained in washing or cleaning agents preferably in amounts of up to 0.15% by weight, in particular from 0.0005% by weight to 0.1% by weight, in each case based on the total agent.

In addition to or in place of the bleach activators and/or the bleach-activating transition metal complexes, use may be made of acylhydrazones of general formula (II)

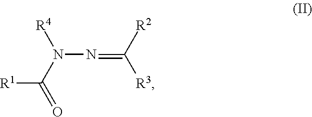

in which R¹ represents a CF₃ group or a C₁₋₂₈ alkyl, C₂₋₂₈ alkenyl, C₂₋₂₂ alkynyl, C₃₋₁₂ cycloalkyl, C₃₋₁₂ cycloalkenyl, phenyl, naphthyl, C₇₋₉ aralkyl, C₃₋₂₀ heteroalkyl or C₃₋₁₂ cycloheteroalkyl group, which may optionally be substituted, R² and R³ independently of one another represent hydrogen or an optionally substituted C₁₋₂₈ alkyl, C₂₋₂₈ alkenyl, C₂₋₂₂ alkynyl, C₃₋₁₂ cycloalkyl, C₃₋₁₂ cycloalkenyl, C₇₋₉ aralkyl, C₃₋₂₈ heteroalkyl, C₃₋₁₂ cycloheteroalkyl, C₅₋₁₆ heteroaralkyl, phenyl, naphthyl or heteroaryl group, or R² and R³ together with the carbon atom connecting them represent an optionally substituted 5-, 6-, 7-, 8- or 9-membered ring which may optionally contain heteroatoms, and R⁴ represents hydrogen or a C₁₋₂₈ alkyl, C₂₋₂₈ alkenyl, C₂₋₂₂ alkynyl, C₃₋₁₂ cycloalkyl, C₃₋₁₂ cycloalkenyl, C₇₋₉ aralkyl, C₃₋₂₀ heteroalkyl, C₃₋₁₂ cycloheteroalkyl, C₅₋₁₆ heteroaralkyl group or an optionally substituted phenyl or naphthyl or heteroaryl group.

The acylhydrazones may be present in an E- or Z-configuration; if R² is hydrogen, the compound of general formula (II) may be present in one of its tautomeric forms or as a mixture thereof.

In the compounds of general formula (II), R² is preferably hydrogen. R¹ and/or R³ is preferably an alkyl, phenyl or naphthyl group substituted with an electron-withdrawing group. R⁴ is preferably hydrogen. A suitable electron-withdrawing group is preferably an ammonium group which optionally carries alkyl or hydroxyalkyl groups or is formed, with inclusion of the N atom carrying an alkyl group, as a heterocycloalkyl group optionally carrying further heteroatoms.

Preferred embodiments of the compounds according to general formula (II) include those of general formula (III)

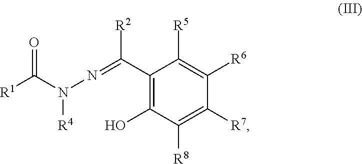

in which R¹ denotes a C₁₋₄ alkyl group which carries a substituent selected from

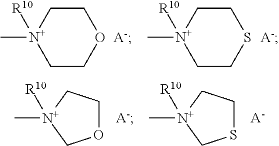

in which R¹⁰ represents hydrogen or a C₁₋₂₈ alkyl, C₂₋₂₈ alkenyl, C₂₋₂₂ alkynyl, C₃₋₁₂ cycloalkyl, C₃₋₁₂ cycloalkenyl, C₇₋₉ aralkyl, C₃₋₂₀ heteroalkyl, C₃₋₁₂ cycloheteroalkyl, C₅₋₁₆ heteroaralkyl group, and A⁻ represents the anion of an organic or inorganic acid, R² and R⁴ have the meaning specified for formula (I), and R⁵, R⁶, R⁷ and R⁸ independently of one another represent R′, hydrogen, halogen, a hydroxyl, amino, optionally substituted N-mono- or -di-C₁₋₄ alkylamino or —C₂₋₄ hydroxyalkylamino, N-phenylamino or N-naphthylamino, C₁₋₂₈ alkyl, C₁₋₂₈ alkoxy, phenoxy, C₂₋₂₈ alkenyl, C₂₋₂₂ alkynyl, C₃₋₁₂ cycloalkyl, C₃₋₁₂ cycloalkenyl, C₇₋₉ aralkyl, C₃₋₂₀ heteroalkyl, C₃₋₁₂ cycloheteroalkyl, C₅₋₁₆ heteroaralkyl, phenyl or naphthyl group, wherein the substituents are selected from C₁₋₄ alkyl, C₁₋₄ alkoxy, hydroxyl, sulfo, sulfato, halogen, cyano, nitro, carboxy, phenyl, phenoxy, naphthoxy, amino, N-mono- or -di-C₁₋₄ alkylamino or —C₂₋₄ hydroxyalkylamino, N-phenylamino or N-naphthylamino groups, or R⁵ and R⁶ or R⁶ and R⁷ or R⁷ and R⁸ are connected to one another to form 1, 2 or 3 carbocyclic or O-, NR¹⁰-, or S-heterocyclic, optionally aromatic and/or optionally C₁₋₆ alkyl-substituted rings.

The anion A⁻ is preferably a carboxylate such as lactate, citrate, tartrate or succinate, perchlorate, tetrafluoroborate, hexafluorophosphate, alkylsulfonate, arylsulfonate such as for example p-toluenesulfonate, alkyl sulfate such as for example methosulfate, hydrogen sulfate, sulfate, dihydrogen phosphate, hydrogen phosphate, phosphate, isocyanate, rhodanide, nitrate, fluoride, chloride, bromide, hydrogen carbonate or carbonate, wherein, in the case of polyvalent anions, the charge compensation can be achieved by the presence of additional cations such as sodium ions or ammonium ions.

Particular preference is given to the acylhydrazone of formula (IV)

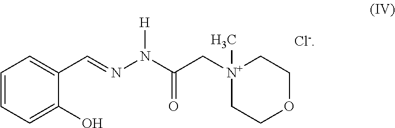 [SMILES: C[N+]1(CC(=O)N/N=C/c2ccccc2O)CCOCC1]

Acylhydrazones of general formulae (II), (III) or of formula (IV) are used in washing or cleaning agents preferably in amounts of 0.001% by weight to 5% by weight, in particular 0.05% by weight to 0.15% by weight.

In one preferred embodiment, an agent according to the invention or used in the context of the use according to the invention or of the method according to the invention contains synthetic anionic surfactant of the sulfate and/or sulfonate type, in particular alkylbenzenesulfonate, fatty alkyl sulfate, fatty alkyl ether sulfate, alkyl and/or dialkyl sulfosuccinate, sulfo fatty acid esters and/or sulfo fatty acid disalts, in particular in an amount ranging from 2% by weight to 25% by weight. Preferably, the anionic surfactant is selected from alkylbenzenesulfonates, alkyl or alkenyl sulfates and/or alkyl or alkenyl ether sulfates in which the alkyl or alkenyl group possesses 8 to 22, in particular 12 to 18 C atoms. These are usually not individual substances but rather cuts or mixtures. Among these, preference is given to those with a proportion of more than 20% by weight compounds having longer-chain radicals in the range from 16 to 18 C atoms.

A further embodiment of such agents encompasses the presence of nonionic surfactant selected from fatty alkylpolyglycosides, fatty alkylpolyalkoxylates, in particular -ethoxylates and/or -propoxylates, fatty acid polyhydroxyamides and/or ethoxylation and/or propoxylation products of fatty alkyl amines, vicinal diols, fatty acid alkyl esters and/or fatty acid amides, as well as mixtures thereof, in particular in an amount ranging from 2% by weight to 25% by weight.

Suitable nonionic surfactants include the alkoxylates, in particular ethoxylates and/or propoxylates, of saturated or mono- to polyunsaturated linear or branched-chain alcohols having 10 to 22 C atoms, preferably 12 to 18 C atoms. The degree of alkoxylation of the alcohols is generally between 1 and 20, preferably between 3 and 10. They can be prepared in a known manner by reacting the corresponding alcohols with the corresponding alkylene oxides. The derivatives of fatty alcohols are particularly suitable, although the branched-chain isomers thereof, in particular so-called oxo alcohols, can also be used to prepare usable alkoxylates. The alkoxylates, in particular the ethoxylates, of primary alcohols having linear, in particular dodecyl, tetradecyl, hexadecyl or octadecyl radicals, as well as mixtures thereof, can accordingly be used. It is also possible to use corresponding alkoxylation products of alkylamines, vicinal diols and carboxylic acid amides, which correspond to the aforementioned alcohols in terms of the alkyl moiety. Also suitable are the ethylene oxide and/or propylene oxide insertion products of fatty acid alkyl esters, as well as fatty acid polyhydroxyamides. So-called alkylpolyglycosides which are suitable for incorporation in the agents used in connection with the invention are compounds of general formula (G)ₙ-OR¹², in which R¹² denotes an alkyl or alkenyl radical having 8 to 22 C atoms, G denotes a glycose unit and n denotes a number between 1 and 10. The glycoside component (G)ₙ denotes oligomers or polymers from naturally occurring aldose or ketose monomers, including in particular glucose, mannose, fructose, galactose, talose, gulose, altrose, allose, idose, ribose, arabinose, xylose and lyxose. The oligomers consisting of such glycosidically linked monomers are characterized not only by the nature of the sugars contained therein but also by the number thereof (the so-called degree of oligomerization). The degree of oligomerization n, as a parameter to be determined analytically, generally assumes fractional numerical values; the value thereof is between 1 and 10, and for the glycosides used with preference is below a value of 1.5, in particular between 1.2 and 1.4. Glucose is a preferred monomer building block on account of its good availability. The alkyl or alkenyl moiety R¹² of the glycosides preferably likewise derives from easily accessible derivatives of renewable raw materials, in particular from fatty alcohols, although the branched-chain isomers thereof, in particular so-called oxo alcohols, can also be used to prepare usable glycosides. The primary alcohols having linear octyl, decyl, dodecyl, tetradecyl, hexadecyl or octadecyl radicals, as well as mixtures thereof, can accordingly be used in particular. Particularly preferred alkylglycosides contain a coconut fatty alkyl radical, that is to say mixtures where substantially R¹²=dodecyl and R¹²=tetradecyl.

In agents which contain an active substance used according to the invention or which are used in the context of the use according to the invention or of the method according to the invention, nonionic surfactant is contained therein preferably in amounts of 1% by weight to 30% by weight, in particular 1% by weight to 25% by weight, wherein amounts in the upper part of this range are more likely to be encountered in liquid washing agents, and washing agents in particle form preferably contain rather smaller amounts of up to 5% by weight.

The agents may instead or additionally contain further surfactants, preferably synthetic anionic surfactants of the sulfate or sulfonate type, including for example the aforementioned alkylbenzenesulfonates, in amounts of preferably no more than 20% by weight, in particular from 0.1% by weight to 18% by weight, in each case based on the total agent. As synthetic anionic surfactants which are particularly suitable for use in such agents, mention may be made of alkyl and/or alkenyl sulfates having 8 to 22 C atoms which carry an alkali-, ammonium- or alkyl- or hydroxyalkyl-substituted ammonium ion as counter-cation. Preference is given to the derivatives of fatty alcohols having in particular 12 to 18 C atoms, and to branched-chain analogs thereof which are known as oxo alcohols. Alkyl sulfates and alkenyl sulfates can be prepared in a known manner by reacting the corresponding alcohol component with a customary sulfating reagent, in particular sulfur trioxide or chlorosulfonic acid, followed by neutralization with alkali-, ammonium- or alkyl- or hydroxyalkyl-substituted ammonium bases. The surfactants of the sulfate type which can be used also include sulfated alkoxylation products of the aforementioned alcohols, which are known as ether sulfates. Such ether sulfates contain preferably 2 to 30, in particular 4 to 10 ethylene glycol groups per molecule. Suitable anionic surfactants of the sulfonate type include the α-sulfo esters obtainable by reacting fatty acid esters with sulfur trioxide and subsequent neutralization, in particular the sulfonation products deriving from fatty acids having 8 to 22 C atoms, preferably 12 to 18 C atoms, and linear alcohols having 1 to 6 C atoms, preferably 1 to 4 C atoms, as well as the sulfo fatty acids stemming therefrom by formal saponification. Preferred anionic surfactants are also the salts of sulfosuccinic acid esters, which are also referred to as alkylsulfosuccinates or dialkylsulfosuccinates and represent the monoesters or diesters of sulfosuccinic acid with alcohols, preferably fatty alcohols and in particular ethoxylated fatty alcohols. Preferred sulfosuccinates contain C₈ to C₁₈ fatty alcohol radicals or mixtures thereof. Particularly preferred sulfosuccinates contain an ethoxylated fatty alcohol radical which, considered per se, represents a nonionic surfactant. Particular preference is thus in turn given to sulfosuccinates having fatty alcohol radicals which derive from ethoxylated fatty alcohols having a restricted homolog distribution.

Soaps may be considered as further optional surfactant-type ingredients, suitable soaps being saturated fatty acid soaps, such as the salts of lauric acid, myristic acid, palmitic acid or stearic acid, and soaps derived from natural fatty acid mixtures, for example coconut, palm kernel, or tallow fatty acids. Particular preference is given to those soap mixtures which are composed of 50% by weight to 100% by weight saturated C₁₂-C₁₈ fatty acid soaps and up to 50% by weight oleic acid soap. Soap is preferably contained in amounts of 0.1% by weight to 5% by weight. However, larger amounts of soap of generally up to 20% by weight may also be contained particularly in liquid agents.

If desired, the agents may also contain betaines and/or cationic surfactants which, if present, are used preferably in amounts of 0.5% by weight to 7% by weight.

In a further embodiment, the agent contains water-soluble and/or water-insoluble builders, in particular selected from alkali aluminosilicate, crystalline alkali silicate having a modulus greater than 1, monomeric polycarboxylate, polymeric polycarboxylate and mixtures thereof, in particular in amounts ranging from 2.5% by weight to 60% by weight.

The agent contains preferably 20% by weight to 55% by weight water-soluble and/or water-insoluble, organic and/or inorganic builders. Water-soluble organic builder substances include in particular those from the class of polycarboxylic acids, in particular citric acid and sugar acids, as well as polymeric (poly)carboxylic acids, in particular polycarboxylates obtainable by oxidation of polysaccharides, polymeric acrylic acids, methacrylic acids, maleic acids and mixed polymers thereof, which may also contain, polymerized into them, small proportions of polymerizable substances having no carboxylic acid functionality. The relative molecular weight of the homopolymers of unsaturated carboxylic acids is generally between 5000 g/mol and 200,000 g/mol, that of the copolymers between 2000 g/mol and 200,000 g/mol, preferably 50,000 g/mol to 120,000 g/mol, based on free acid. A particularly preferred acrylic acid/maleic acid copolymer has a relative molecular weight of 50,000 g/mol to 100,000 g/mol. Compounds of this class which are suitable but less preferred are copolymers of acrylic acid or methacrylic acid with vinyl ethers, such as vinyl methyl ethers, vinyl esters, ethylene, propylene and styrene, in which the proportion of acid is at least 50% by weight. As water-soluble organic builder substances, use may also be made of terpolymers which contain two carboxylic acids and/or salts thereof as monomers and, as a third monomer, vinyl alcohol and/or a vinyl alcohol derivative or a carbohydrate. The first acidic monomer or salt thereof is derived from an ethylenically monounsaturated C₃-C₈ carboxylic acid and preferably from a C₃-C₄ monocarboxylic acid, in particular from (meth)acrylic acid. The second acidic monomer or salt thereof may be a derivative of a C₄-C₈ dicarboxylic acid, particular preference being given to maleic acid. The third monomeric unit is in this case formed by vinyl alcohol and/or preferably an esterified vinyl alcohol. Particular preference is given to vinyl alcohol derivatives which represent an ester of short-chain carboxylic acids, for example of C₁-C₄ carboxylic acids, with vinyl alcohol. Preferred terpolymers contain 60% by weight to 95% by weight, in particular 70% by weight to 90% by weight (meth)acrylic acid and/or (meth)acrylate, particularly preferably acrylic acid and/or acrylate, and maleic acid and/or maleinate, as well as 5% by weight to 40% by weight, preferably 10% by weight to 30% by weight vinyl alcohol and/or vinyl acetate. Very particular preference is given to terpolymers in which the weight ratio of (meth)acrylic acid and/or (meth)acrylate to maleic acid and/or maleate is between 1:1 and 4:1, preferably between 2:1 and 3:1 and in particular 2:1 and 2.5:1. Both the amounts and the weight ratios are based on the acids. The second acidic monomer or salt thereof may also be a derivative of an allylsulfonic acid which is substituted in the 2-position with an alkyl radical, preferably with a C₁-C₄ alkyl radical, or with an aromatic radical which is derived preferably from benzene or benzene derivatives. Preferred terpolymers contain 40% by weight to 60% by weight, in particular 45 to 55% by weight (meth)acrylic acid and/or (meth)acrylate, particularly preferably acrylic acid and/or acrylate, 10% by weight to 30% by weight, preferably 15% by weight to 25% by weight methallylsulfonic acid and/or methallylsulfonate, and as a third monomer 15% by weight to 40% by weight, preferably 20% by weight to 40% by weight of a carbohydrate. This carbohydrate may be for example a mono-, di-, oligo- or polysaccharide, preference being given to mono-, di- or oligosaccharides and particular preference being given to sucrose. The use of the third monomer is presumed to incorporate defined break points into the polymer, which are responsible for the good biodegradability of the polymer. These terpolymers generally have a relative molecular weight between 1000 g/mol and 200,000 g/mol, preferably between 2000 g/mol and 50,000 g/mol, and in particular between 3000 g/mol and 10,000 g/mol. Particularly for the manufacture of liquid agents, they can be used in the form of aqueous solutions, preferably in the form of 30 to 50% by weight aqueous solutions. All the aforementioned polycarboxylic acids are generally used in the form of their water-soluble salts, in particular their alkali salts.

Such organic builder substances are contained preferably in amounts of up to 40% by weight, in particular up to 25% by weight and particularly preferably from 1% by weight to 5% by weight. Amounts close to the aforementioned upper limit are preferably used in paste-like or liquid agents, particularly water-containing agents.

As water-insoluble, water-dispersible inorganic builder materials, use is made in particular of crystalline or amorphous alkali aluminosilicates, in amounts of up to 50% by weight, preferably not more than 40% by weight, and in liquid agents in particular from 1% by weight to 5% by weight. Among these, preference is given to the crystalline aluminosilicates of washing agent quality, in particular zeolite NaA and optionally NaX. Amounts close to the aforementioned upper limit are used preferably in solid, particulate agents. Suitable aluminosilicates have in particular no particles with a particle size greater than 30 μm, and preferably are made up of at least 80% by weight particles having a size less than 10 μm. Their calcium binding capability, which can be determined as indicated in German patent DE 2412837, is in the range from 100 to 200 mg CaO per gram. Suitable substitutes or partial substitutes for the aforementioned aluminosilicate are crystalline alkali silicates, which may be present alone or in a mixture with amorphous silicates. The alkali silicates which can be used as builders in the agents preferably have a molar ratio of alkali oxide to SiO₂ below 0.95, in particular from 1:1.1 to 1:12, and may be present in amorphous or crystalline form. Preferred alkali silicates are sodium silicates, in particular amorphous sodium silicates, having a molar ratio Na₂O:SiO₂ of from 1:2 to 1:2.8. Such amorphous alkali silicates are commercially available for example under the name Portil®. Those having a molar ratio Na₂O:SiO₂ of from 1:1.9 to 1:2.8 are added, during manufacture, preferably as a solid and not in the form of a solution. As crystalline silicates which may be present alone or in a mixture with amorphous silicates, use is preferably made of crystalline phyllosilicates of general formula Na₂SiₓO₂ₓ₊₁.yH₂O, in which x, the so-called modulus, is a number from 1.9 to 4 and y is a number from 0 to 20, and preferred values for x are 2, 3 or 4. Preferred crystalline phyllosilicates are those in which x in the aforementioned general formula assumes the values 2 or 3. In particular, preference is given to both β- and δ-sodium disilicates (Na₂Si₂O₅.yH₂O). Practically anhydrous crystalline alkali silicates which are prepared from amorphous alkali silicates and have the aforementioned general formula, in which x denotes a number from 1.9 to 2.1, can also be used in agents which contain an active substance to be used according to the invention. In a further preferred embodiment of agents according to the invention, a crystalline sodium phyllosilicate having a modulus of 2 to 3 is used, such as the one that can be prepared from sand and soda. Crystalline sodium silicates having a modulus in the range from 1.9 to 3.5 are used in a further preferred embodiment of washing agents which contain an active substance used according to the invention. The alkali silicate content thereof is preferably 1% by weight to 50% by weight and in particular 5% by weight to 35% by weight, based on anhydrous active substance. If alkali aluminosilicate, in particular zeolite, is also present as an additional builder substance, the alkali silicate content is preferably 1% by weight to 15% by weight and in particular 2% by weight to 8% by weight, based on anhydrous active substance. The weight ratio of aluminosilicate to silicate, in each case based on anhydrous active substances, is then preferably 4:1 to 10:1. In agents which contain both amorphous and crystalline alkali silicates, the weight ratio of amorphous alkali silicate to crystalline alkali silicate is preferably 1:2 to 2:1 and in particular 1:1 to 2:1.

In addition to the aforementioned inorganic builders, further water-soluble or water-insoluble inorganic substances may be contained in the agents which contain an active substance to be used according to the invention, are used together therewith or are used in the method according to the invention. Alkali carbonates, alkali hydrogen carbonates and alkali sulfates, as well as mixtures thereof, are suitable in this connection. Such additional inorganic material may be present in amounts of up to 70% by weight.

In addition, the agents may contain further constituents which are customary in washing or cleaning agents. These optional constituents include in particular enzymes, enzyme stabilizers, complexing agents for heavy metals, for example aminopolycarboxylic acids, aminohydroxypolycarboxylic acids, polyphosphonic acids and/or aminopolyphosphonic acids, foam inhibitors, for example organopolysiloxanes or paraffins, solvents and optical brighteners, for example stilbenedisulfonic acid derivatives. Up to 1% by weight, in particular 0.01% by weight to 0.5% by weight optical brighteners, in particular compounds from the class of substituted 4,4′-bis-(2,4,6-triamino-s-triazinyl)stilbene-2,2′-disulfonic acids, up to 5% by weight, in particular 0.1% by weight to 2% by weight complexing agents for heavy metals, in particular aminoalkylenephosphonic acids and salts thereof, and up to 2% by weight, in particular 0.1% by weight to 1% by weight foam inhibitors, are preferably contained in agents which contain an active substance used according to the invention, the aforementioned proportions by weight being based in each case on the total agent.

Solvents which may be used in particular in liquid agents are, besides water, preferably those which are miscible with water. These include the lower alcohols, for example ethanol, propanol, isopropanol, and the isomeric butanols, glycerol, lower glycols, for example ethylene glycol and propylene glycol, and the ethers derivable from the aforementioned classes of compounds. In such liquid agents, the active substances used according to the invention are generally present in dissolved or suspended form.

The enzymes which are preferably present are selected in particular from the group comprising protease, amylase, lipase, cellulase, hemicellulase, oxidase, peroxidase, pectinase and mixtures thereof. Protease obtained from microorganisms, such as bacteria or fungi, is especially suitable. It can be obtained in a known manner from suitable microorganisms by fermentation processes. Proteases are commercially available for example under the names BLAP®, Savinase®, Esperase®, Maxatase®, Optimase®, Alcalase®, Durazym® or Maxapem®. The lipase which can be used can be obtained for example from Humicola lanuginosa, from Bacillus species, from Pseudomonas species, from Fusarium species, from Rhizopus species or from Aspergillus species. Suitable lipases are commercially available for example under the names Lipolase®, Lipozym®, Lipomax®, Lipex®, Amano® Lipase, Toyo-Jozo® Lipase, Meito® Lipase and Diosynth® Lipase. Suitable amylases are commercially available for example under the names Maxamyl®, Termamyl®, Duramyl® and Purafect® OxAm. The cellulase which can be used may be an enzyme which can be obtained from bacteria or fungi and which has a pH optimum preferably in the weakly acidic to weakly alkaline range from 6 to 9.5. Such cellulases are commercially available under the names Celluzyme®, Carezyme® and Ecostone®. Suitable pectinases are available for example under the names Gamanase®, Pektinex AR®, X-Pect® or Pectaway® from Novozymes, under the name Rohapect UF®, Rohapect TPL®, Rohapect PTE100®, Rohapect MPE®, Rohapect MA plus HC, Rohapect DA12L®, Rohapect 10L®, Rohapect® B1L from AB Enzymes, and under the name Pyrolase® from Diversa Corp., San Diego, Calif., USA.

The customary enzyme stabilizers which are optionally present particularly in liquid agents include aminoalcohols, for example mono-, di- and triethanolamine and propanolamine, and mixtures thereof, lower carboxylic acids, boric acid, alkali borates, boric acid/carboxylic acid combinations, boric acid esters, boronic acid derivatives, calcium salts, for example calcium/formic acid combination, magnesium salts, and/or sulfur-containing reducing agents.

Suitable foam inhibitors include long-chain soaps, in particular behenic soap, fatty acid amides, paraffins, waxes, microcrystalline waxes, organopolysiloxanes, and mixtures thereof, which may additionally contain microfine, optionally silanized or otherwise hydrophobicized silicic acid. For use in particulate agents, such foam inhibitors are preferably bound to granular, water-soluble carrier substances.

The soil-release-enabling polymers known to be polyester-active, which may be used in addition to the active substances essential to the invention, include copolyesters of dicarboxylic acids, for example adipic acid, phthalic acid or terephthalic acid, diols, for example ethylene glycol or propylene glycol, and polydiols, for example polyethylene glycol or polypropylene glycol. The soil-release-enabling polyesters which are preferably used include those compounds which are accessible formally by esterification of two monomer parts, the first monomer being a dicarboxylic acid HOOC-Ph-COOH and the second monomer being a diol HO—(CHR¹¹—)ₐOH, which may also be present as a polymeric diol H—(O—(CHR¹¹)ₐ)_{b}OH. Therein, Ph denotes an o-, m- or p-phenylene radical which may carry 1 to 4 substituents selected from alkyl radicals having 1 to 22 C atoms, sulfonic acid groups, carboxyl groups, and mixtures thereof, R¹¹ denotes hydrogen, an alkyl radical having 1 to 22 C atoms, and mixtures thereof, a is a number from 2 to 6, and b is a number from 1 to 300. Preferably, both monomer diol units —O—(CHR¹¹—)ₐO— and polymer diol units —(O—(CHR¹¹)ₐ)_{b}O— are present in the polyesters obtainable from these. The molar ratio of monomer diol units to polymer diol units is preferably 100:1 to 1:100, in particular 10:1 to 1:10. In the polymer diol units, the degree of polymerization b is preferably in the range from 4 to 200, in particular from 12 to 140. The molecular weight or the average molecular weight or the maximum of the molecular weight distribution of preferred soil-release-enabling polyesters is in the range from 250 to 100,000, in particular from 500 to 50,000. The acid on which the Ph radical is based is selected preferably from terephthalic acid, isophthalic acid, phthalic acid, trimellitic acid, mellitic acid, the isomers of sulfophthalic acid, sulfoisophthalic acid and sulfoterephthalic acid, and mixtures thereof. If the acid groups thereof are not part of the ester bonds in the polymer, they are preferably present in salt form, in particular as an alkali salt or ammonium salt. Among these, particular preference is given to the sodium and potassium salts. If desired, instead of the monomer HOOC-Ph-COOH, small proportions, in particular no more than 10 mol % based on the proportion of Ph having the meaning indicated above, of other acids which have at least two carboxyl groups may be contained in the soil-release-enabling polyester. These include for example alkylene- and alkenylenedicarboxylic acids such as malonic acid, succinic acid, fumaric acid, maleic acid, glutaric acid, adipic acid, pimelic acid, suberic acid, azelaic acid and sebacic acid. The preferred diols HO—(CHR¹¹—)ₐOH are those in which R¹¹ is hydrogen and a is a number from 2 to 6, and those in which a has the value 2 and R¹¹ is selected from hydrogen and the alkyl radicals having 1 to 10, in particular 1 to 3 C atoms. Among the last-mentioned diols, particular preference is given to those of formula HO—CH₂CHR¹¹—OH, in which R¹¹ has the aforementioned meaning. Examples of diol components are ethylene glycol, 1,2-propylene glycol, 1,3-propylene glycol, 1,4-butanediol, 1,5-pentanediol, 1,6-hexanediol, 1,8-octanediol, 1,2-decanediol, 1,2-dodecanediol and neopentyl glycol. Among the polymeric diols, particular preference is given to polyethylene glycol having an average molecular weight in the range from 1000 g/mol to 6000 g/mol.

If desired, these polyesters having the composition described above may also be closed by end groups, wherein suitable end groups are alkyl groups having 1 to 22 C atoms and esters of monocarboxylic acids. The end groups bound via ester bonds may be based on alkyl-, alkenyl- and arylmonocarboxylic acids having 5 to 32 C atoms, in particular 5 to 18 C atoms. These include valeric acid, caproic acid, oenanthic acid, caprylic acid, pelargonic acid, capric acid, undecanoic acid, undecenoic acid, lauric acid, lauroleic acid, tridecanoic acid, myristic acid, myristoleic acid, pentadecanoic acid, palmitic acid, stearic acid, petroselic acid, petroselaidic acid, oleic acid, linoleic acid, linolaidic acid, linolenic acid, eleostearic acid, arachidic acid, gadoleic acid, arachidonic acid, behenic acid, erucic acid, brassidic acid, clupanodonic acid, lignoceric acid, cerotic acid, melissic acid, and benzoic acid which may carry 1 to 5 substituents having a total of up to 25 C atoms, in particular 1 to 12 C atoms, for example tert-butylbenzoic acid. The end groups may also be based on hydroxymonocarboxylic acids having 5 to 22 C atoms, which include for example hydroxyvaleric acid, hydroxycaproic acid, ricinoleic acid, its hydrogenation product hydroxystearic acid, as well as o-, m- and p-hydroxybenzoic acid. The hydroxymonocarboxylic acids may for their part be connected to one another via their hydroxyl group and their carboxyl group, and may thus be present more than once in an end group. The number of hydroxymonocarboxylic acid units per end group, that is to say the degree of oligomerization thereof, is preferably in the range from 1 to 50, in particular from 1 to 10. In a preferred embodiment of the invention, polymers of ethylene terephthalate and polyethylene oxide terephthalate, in which the polyethylene glycol units have molecular weights of 750 g/mol to 5000 g/mol and the molar ratio of ethylene terephthalate to polyethylene oxide terephthalate is 50:50 to 90:10, are used in combination with an active substance essential to the invention.

The soil-release-enabling polymers are preferably water-soluble, the term “water-soluble” being intended to mean a solubility of at least 0.01 g, preferably at least 0.1 g, of the polymer per liter of water at room temperature and pH 8. Polymers which are used with preference nevertheless have under these conditions a solubility of at least 1 g per liter, in particular at least 10 g per liter.

The preparation of solid agents according to the invention presents no difficulties and can take place in a known manner, for example by spray drying or granulation, wherein enzymes and any further heat-sensitive ingredients, such as for example bleaching agents, are, if applicable, added separately later. In order to prepare agents according to the invention with an increased bulk weight, in particular in the range from 650 g/l to 950 g/l, preference is given to a method involving an extrusion step.

In order to prepare agents according to the invention in the form of tablets, which can be single-phase or multi-phase, single-colored or multi-colored, and in particular can be made up of one layer or of multiple layers, in particular two layers, it is preferable to proceed in such a way that all the constituents (of each layer, if applicable) are mixed together in a mixer and the mixture is compressed by means of conventional tablet presses, for example eccentric presses or rotary presses, at compression pressures in the range from approximately 50 to 100 kN, preferably at 60 to 70 kN. Particularly in the case of multi-layer tablets, it may be advantageous if at least one layer is pre-compressed. This is carried out preferably at compression pressures between 5 and 20 kN, in particular at 10 to 15 kN. Break-resistant tablets which are nevertheless sufficiently rapidly soluble under use conditions, having fracture strength and flexural strength values normally from 100 to 200 N but preferably above 150 N, are thus obtained without difficulty. A tablet manufactured in this way preferably has a weight of 10 g to 50 g, in particular 15 g to 40 g. The tablets may have any three-dimensional shape and may be round, oval or polygonal, wherein intermediate shapes are also possible. Corners and edges are advantageously rounded. Round tablets preferably have a diameter of 30 mm to 40 mm. In particular, the size of polygonal or cuboidal tablets, which are introduced predominantly via the dispensing apparatus for example of the dishwasher, depends on the geometry and volume of said dispensing apparatus. Embodiments which are preferred by way of example have a base area of (20 to 30 mm)×(34 to 40 mm), in particular 26×36 mm or 24×38 mm.

Liquid or paste-like agents according to the invention in the form of solutions containing customary solvents, in particular water, are generally manufactured by simple mixing of the ingredients, which can be introduced in substance or as a solution into an automatic mixer.

In a preferred embodiment, an agent into which active substance to be used according to the invention is incorporated is liquid and contains 1% by weight to 15% by weight, in particular 2% by weight to 10% by weight nonionic surfactant, 2% by weight to 30% by weight, in particular 5% by weight to 20% by weight synthetic anionic surfactant, up to 15% by weight, in particular 2% by weight to 12.5% by weight soap, 0.5% by weight to 5% by weight, in particular 1% by weight to 4% by weight organic builder, in particular polycarboxylate such as citrate, up to 1.5% by weight, in particular 0.1% by weight to 1% by weight complexing agent for heavy metals, such as phosphonate and, besides optionally contained enzyme, enzyme stabilizer, dye and/or fragrance, also water and/or water-miscible solvent.

In a further preferred embodiment, an agent into which active substance to be used according to the present invention is incorporated is particulate and contains up to 25% by weight, in particular 5% by weight to 20% by weight bleaching agent, in particular alkali percarbonate, up to 15% by weight, in particular 1% by weight to 10% by weight bleach activator, 20% by weight to 55% by weight inorganic builder, up to 10% by weight, in particular 2% by weight to 8% by weight water-soluble organic builder, 10% by weight to 25% by weight synthetic anionic surfactant, 1% by weight to 5% by weight nonionic surfactant, and up to 25% by weight, in particular 0.1% by weight to 25% by weight inorganic salts, in particular alkali carbonate and/or alkali hydrogen carbonate.

### Examples

### Example 1: Synthesis

### a) Preparation of 5,11,17,23-tetrasulfo-25,26,27,28-tetrapropoxycalix[4]arene (B1)

Sulfocalix[4]arene (700 mg, 0.871 mmol) and NaOH (1.07 g, 26.7 mmol) were dissolved in 7 ml of water with stirring. A solution of 1-iodopropane (2.61 ml, 4.57 g, 26.9 mmol) in 27 ml of dimethylsulfoxide was then added dropwise. The reaction mixture was stirred for 1 day at 50° C., resulting in the precipitation of a colorless solid. After cooling to room temperature, the mixture was diluted with methanol (40 ml); further raw product precipitated out. The solid obtained was suspended in 2.33 ml of water and filtered off again. Ethanol was added to the filtrate until a colorless solid precipitated out, which was separated off by filtration. This procedure was repeated 3 times in order to remove NaI. The colorless product was dried at 80° C. in a high vacuum.

Yield: 390 mg (0.390 mmol, 45%); colorless solid.

Melting point: >331° C. (decomposition).

IR: 2961 (m) (C—H); 2933 (m) (C—H); 2874 (m) (C—H); 2358 (w), 1632 (w), 1590 (w), 1460 (m) (C—H); 1415 (w), 1385 (w) (C—H); 1263 (m), 1180 (s) (S═O), 1119 (s), 1052 (s) (S═O); 1006 (m), 963 (m), 887 (m) (Ar—H); 806 (m), 806 (m), 700 (m), 664 (s), 620 (s), 592 (m), 560 (m), 513 (m), 486 (m), 441 (m), 405 (m).

¹H-NMR (400.13 MHz, DMSO-d₆): δ_{H}=0.95 (12H, t, J=7.4 Hz, CH₃), 1.92 (8H, sext, J=7.6 Hz, CH₂), 3.25 (4H, d, J=12.8 Hz, ArCH₂Ar), 3.85 (8H, t, J=7.6 Hz, OCH₂), 4.36 (4H, d, J=12.4 Hz, ArCH₂Ar), 7.12 (8H, s, Ar—H).

¹³C-NMR (100.62 MHz, DMSO-d₆): δ_{C}=10.08 (CH₃), 22.60 (CH₂), 33.70 (ArCH₂Ar), 76.53 (OCH₂), 125.91 (ArC—H), 133.22 (ArC—CH₂), 141.56 (ArC—SO₃), 156.38 (ArC—O).

MS (MALDI-TOF, dhb): calculated for C₄₀H₄₄Na₄O₁₆S₄: 1000.1; found: m/z=979 [(M+2H—Na)⁺], 1001 [(M+H)⁺].

### b) Preparation of 5,11,17,23,29,35,41,47-octasulfo-49,50,51,52,53,54,55,56-octaoctoxycalix[8]arene (B2)

Sulfocalix[8]arene (2.50 g, 1.50 mmol) and NaOH (2.45 mg, 61.2 mmol) were dissolved in 15.6 ml of water with stirring. A solution of 1-iodooctane (11.3 ml, 15.1 g, 61.5 mmol) in 62.5 ml of dimethylsulfoxide was then added dropwise. The reaction mixture was stirred for 1 day at 80° C., resulting in the precipitation of a colorless solid. After cooling to room temperature, the mixture was diluted with methanol (50 ml); further raw product precipitated out. The colorless solid obtained was suspended in 20 ml of water and filtered off again. Ethanol was added to the filtrate until a colorless solid precipitated out, which was separated off by filtration. This procedure was repeated 3 times in order to remove NaI.

Yield: 1.42 g (0.555 mmol, 37%); colorless solid.

Melting point: >316° C. (decomposition).

IR: 3443, 2954 (m) (C—H); 2923 (m) (C—H); 2853 (m) (C—H); 2357 (w), 2194 (w), 2015 (w), 1635 (w), 1591 (w), 1454 (m) (C—H); 1412 (w), 1382 (w) (C—H); 1260 (m), 1174 (s), 1138 (s), 1051 (s) (S═O); 997 (m), 960 (w), 897 (w) (Ar—H); 721 (w), 671 (s), 620 (s), 591 (s), 537 (s), 428 (m).

¹H-NMR (400.13 MHz, DMSO-d₆): δ_{H}=0.84 (24H, t, CH₃), 1.10-1.55 (80H, br, CH₂), 1.60-1.76 (16H, br, OCH₂CH₂), 3.42-3.92 (24H, m, ArCH₂Ar, OCH₂), 4.27 (8H, br, ArCH₂Ar), 7.25 (16H, s, Ar—H).

¹³C-NMR (100.62 MHz, DMSO-d₆): δ_{C}=13.77 (CH₃), 22.23 (CH₂CH₃), 25.91 (CH₂), 28.62 (CH₂), 29.24 (CH₂), 29.43 (CH₂), 29.72 (CH₂), 31.52 (ArCH₂Ar), 72.78 (OCH₂), 125.93 (ArC—H), 132.33 (ArC—CH₂), 142.52 (ArC—SO₃), 155.35 (ArC—O).

MS (MALDI-TOF, sin): calculated for C₁₂₀H₁₆₈Na₈O₃₂S₈: 2560.8; found: m/z=2563 [(M+2H)⁺], 2562 [(M+H)⁺].

### c) Preparation of 5,11,17,23-tetrasulfo-25,26,27,28-tetraoctoxycalix[4]arene (B3)

Sulfocalix[4]arene (300 mg, 0.374 mmol) and NaOH (614 mg, 15.2 mmol, 10.2 eq.) were dissolved in 3 ml of water with stirring. 1-Iodooctane (1.41 ml, 1.88 g, 7.67 mmol, 5.1 eq.) in 10 ml of dimethylsulfoxide was then added dropwise. The reaction mixture was stirred for 1 day at 90° C., resulting in the precipitation of a colorless solid. After cooling to room temperature, the mixture was diluted with methanol (40 ml) in order to precipitate out further raw product. After filtration, the solid was dissolved in water and the insoluble residue was filtered off. Ethanol was added to the filtrate until a colorless solid precipitated out, which was separated off by filtration. The colorless product was dried at 80° C. in a high vacuum.

Yield: 265 mg (0.207 mmol, 55%); colorless solid.

Melting point: >390° C. (decomposition).

IR: 2921 (w) (C—H); 2852 (w) (C—H); 2017 (w), 1777 (w), 1431 (s) (C—H); 1157 (m) (S═O), 1103 (m), 995 (m), 880 (s) (Ar—H); 779 (m), 701 (m), 666 (m), 618 (m), 559 (m), 439 (m).

¹H-NMR (400.13 MHz, DMSO-d₆): δ_{H}=0.87 (12H, t, J=6.8 Hz CH₃), 1.23-1.43 (40H, br, CH₂), 1.86-1.98 (8H, br, OCH₂CH₂), 3.14 (4H, d, J=13.2 Hz, ArCH₂Ar), 3.87 (8H, t, J=7.2 Hz, OCH₂), 4.33 (4H, d, J=13.2 Hz, ArCH₂Ar), 7.07 (8H, s, Ar—H).

¹³C-NMR (100.62 MHz, DMSO-d₆): δ_{C}=13.83 (CH₃), 22.11 (CH₂CH₃), 25.82 (CH₂), 29.03 (CH₂), 29.44 (CH₂), 29.71 (CH₂), 31.43 (ArCH₂Ar), 66.28 (OCH₂), 125.74 (ArC—H), 133.10 (ArC—CH₂), 141.62 (ArC—SO₃), 156.22 (ArC—O).

MS (MALDI-TOF, sin): calculated for C₆₀H₈₄Na₄O₁₆S₄: 1280.4; found: m/z=1281 [(M+H)⁺].

### Example 2: Washing Tests

Aqueous solutions I (200 mg/l n-dodecylbenzenesulfonate) and II (427 mg/l Na lauryl ether sulfate and 330 mg/l C₁₂₋₁₈ fatty alcohol, 7-tuply ethoxylated, and 340 mg/l n-dodecylbenzenesulfonate) were adjusted to pH 8.5 and were used to wash at 20° C. a white cotton fabric soiled with a standardized tomato-based meat sauce. The same surfactant solutions after the addition of 572 mg/l of the calixarene B2 prepared in example 1 were also adjusted to pH 8.5 and used. After the test fabrics had been hung up to dry and put through a mangle, the brightness value Y thereof was determined spectrophotometrically (Minolta® CR200-1). The brightness values (as mean values from 5 determinations) are indicated in table 1 below.

TABLE 1
Washing results (figures in %)

| Solution | Y |
|---|---|
| I | 75.8 |
| I + B2 | 79.7 |
| II | 75.9 |
| II + B2 | 79.1 |

A washing solution containing the active substance to be used according to the invention exhibited a much better primary detergent performance than a washing solution of otherwise identical composition but without said active substance.

### Example 3

Example 2 was repeated using the calixarene B3 prepared in example 1, but in contrast to example 2 a standardized baby food/pureed carrot stain was used. The brightness values are indicated in table 2 below.

TABLE 2
Washing results (figures in %)

| Solution | Y |
|---|---|
| I | 96.9 |
| I + B3 | 97.3 |
| II | 96.6 |
| II + B3 | 97.2 |

Here, too, the washing solution containing the active substance to be used according to the invention exhibited a much better primary detergent performance than a washing solution of otherwise identical composition but without said active substance.

While at least one exemplary embodiment has been presented in the foregoing detailed description of the invention, it should be appreciated that a vast number of variations exist. It should also be appreciated that the exemplary embodiment or exemplary embodiments are only examples, and are not intended to limit the scope, applicability, or configuration of the invention in any way. Rather, the foregoing detailed description will provide those skilled in the art with a convenient road map for implementing an exemplary embodiment of the invention, it being understood that various changes may be made in the function and arrangement of elements described in an exemplary embodiment without departing from the scope of the invention as set forth in the appended claims and their legal equivalents.

What is claimed is:

1. A washing or cleaning agent, containing a compound of general formula (I) 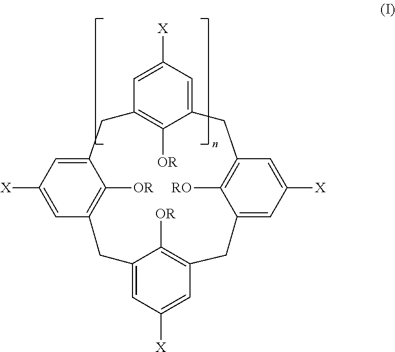
in which n represents a number from 1 to 9, each R independently represents hydrogen or an alkyl group having 1 to 20 C atoms, each X independently represents hydrogen, —Y—COOM or —Y—SO₃M, each Y independently represents a direct bond, an alkylene group having 1 to 12 C atoms, an arylene or heteroarylene group having 2 to 12 C atoms, a mono- or poly-alkyl-substituted arylene group or a mono- or poly-aryl-substituted alkylene group having 6 to 18 C atoms, and M represents hydrogen, an alkali metal atom or an ammonium group, wherein at least 1 substituent X is not hydrogen and that at least 1 substituent R is not hydrogen.

2. The agent according to claim 1, wherein it contains the compound of the general formula (I) in amounts of 0.1% by weight to 10% by weight.

3. The agent according to claim 1, wherein in the compound of general formula (I) n denotes a number from 3 to 7.

4. The agent according to claim 1, wherein in the compound of general formula (I) R denotes hydrogen or an alkyl group having 6 to 12 C atoms.

5. The agent according to claim 1, wherein in the compound of general formula (I) all substituents X are not hydrogen.

6. A method for removing soiling from textiles or hard surfaces, comprising a step wherein the textiles or hard surfaces are contacted with a washing or cleaning agent and a compound of general formula (I) 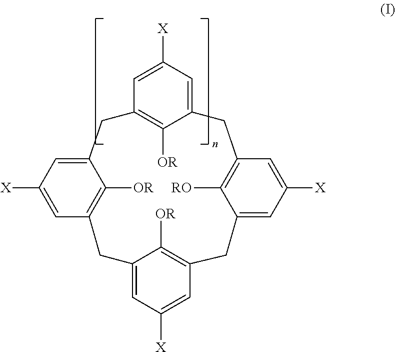
in which n represents a number from 1 to 9, each R independently represents hydrogen or an alkyl group having 1 to 20 C atoms, each X independently represents hydrogen, —Y—COOM or —Y—SO₃M, each Y independently represents a direct bond, an alkylene group having 1 to 12 C atoms, an arylene or heteroarylene group having 2 to 12 C atoms, a mono- or poly-alkyl-substituted arylene group or a mono- or poly-aryl-substituted alkylene group having 6 to 18 C atoms, and M represents hydrogen, an alkali metal atom or an ammonium group, wherein at least 1 substituent X is not hydrogen and that at least 1 substituent R is not hydrogen.

7. The method according to claim 6, wherein the compound of general formula (I) is added to a liquor containing washing or cleaning agent or is introduced into the liquor as a constituent of a washing or cleaning agent.

8. The method according to claim 6, wherein the concentration of the compound of general formula (I) in liquor containing the agent is in the range from 0.1 mmol/l to 2.5 mmol/l.

9. The method according to claim 6, wherein it is carried out by means of a domestic washing machine or dishwasher.
